(19) United States
(12) Patent Application Publication
(10) Pub. No.: US 2003/0236407 A1
(43) Pub. Date: Dec. 25, 2003

(54) Perfluoroalkyl group-containing micelle-forming triiodoaromatic compounds, process for their production and their use as contrast media

(75) Inventors: Markus Schafer, Berlin (DE); Bernd Raduechel, Berlin (DE); Heribert Miklautz, Berlin (DE); Franz Maier, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/375,044

(22) Filed: Feb. 28, 2003

Related U.S. Application Data
(60) Provisional application No. 60/363,879, filed on Mar. 14, 2002.

(30) Foreign Application Priority Data
Feb. 28, 2002 (DE) 10209018.1

(51) Int. Cl. C07D 241/04

(52) U.S. Cl. 544/384

(57) ABSTRACT

The invention relates to new, perfluoroalkyl group-containing triiodoaromatic compounds, process for their production, their use as contrast media for x-ray diagnosis as well as for magnetic resonance diagnosis and magnetic resonance spectroscopy by means of fluorine measurement. Subjects of the invention are also diagnostic agents that contain these new compounds.

This application claims the benefit of the filing date of U.S. Provisional Application Serial No. 60/363,879 filed Mar. 14, 2002.

The invention relates to the subjects that are characterized in the claims, i.e., new perfluoroalkyl group-containing triiodoaromatic compounds, process for their production, their use as contrast media for x-ray diagnosis as well as for magnetic resonance diagnosis and magnetic resonance spectroscopy by means of fluorine measurement. Subjects of the invention are also diagnostic agents that contain these new compounds.

There are a number of iodized organic compounds that can be used as x-ray contrast media since the iodine atom effectively absorbs the x-ray radiation. Such x-ray contrast media are described in, for example, U.S. Pat. Nos. 2,786,055, US 3,795,698, US 4,735,795 and US 5,047,228. Iodine-containing x-ray contrast media, which are especially suitable for visualization of vessels, are also described in DE 43 44 464 A1. The compounds that are described in DE 43 44 464 A1 are iodine-containing dendritic polymers that have a nitrogen-containing nucleus and triiodoaromatic compounds that contain imaging radicals.

In addition, x-ray contrast media that contain iodoaromatic compounds for study of the gastrointestinal tract are described in U.S. Pat. Nos. 5,422,114 and 5,326,553. Example 4 in U.S. Pat. No. 5,326,553 discloses as x-ray contrast medium the compound (2,4,6-triiodophenoxy)-1H,1H,2H,2H-perfluorooctane, in which there is a perfluoroalkyl group-containing triiodoaromatic compound.

WO 94/05335 discloses triiodoaromatic compounds with a —COCF₃ group as a group that produces nuclear magnetic resonance, in particular N-trifluoroacetyl-iopanoic acid and fluoro-N-trifluoroacetyl-iopanoic acid. The compounds are described both as x-ray contrast media and as contrast media for ¹⁹F-imaging. WO 99/02193 describes perfluorine group-containing triiodoaromatic compounds that are suitable for x-ray diagnosis.

It has been shown, however, that the contrast media of the prior art cannot be used satisfactorily for all applications. The object of the invention is therefore to find new contrast media that are very well suited both for x-ray diagnosis and for magnetic resonance diagnosis by means of fluorine measurement, in particular for magnetic resonance tomography.

It has now been found, surprisingly enough, that the compounds in aqueous solution according to the invention form micelles at very low concentrations (small “critical micelle forming concentration,” CMC). This property essentially influences the distribution and kinetics after administration in the test animal. In addition, the compounds according to the invention have excellent water solubility.

The solution of the object of the invention is achieved with the perfluoroalkyl group-containing triiodoaromatic compounds of general formula I.

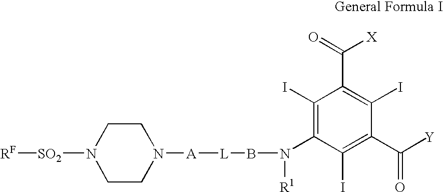

R^{F} means a straight-chain or branched carbon chain with formula —CₙF₂ₙG, in which G stands for a terminal fluorine, chlorine, iodine or hydrogen atom, and n stands for numbers 4-30.

A and B are either like or unlike groupings and mean a carbonyl group or sulfonyl group. L means a straight-chain, branched, saturated or unsaturated carbon chain with 1-15 C atoms, which can be interrupted by 1-5 oxygen atoms, 1-3 sulfur atoms, or 1-5 sulfonyl groups, and the latter can optionally be substituted with 1-6 hydroxy groups or 1-3-(CH₂)ₚ—CO₂H groups, whereby p stands for numbers from 0-10.

R¹ means a hydrogen atom or the group —(CH₂)_{q}—CO₂H, whereby q stands for numbers from 1-10.

In addition,

X represents OH, —O⁻Na⁺, —O⁻meglumine⁺ or NR², R³,

Y represents OH, —O⁻Na⁺, —O⁻meglumine⁺ or NR⁴R⁵,

whereby

R², R³, R⁴, R⁵ can be the same or different from one another, and represent hydrogen, a straight-chain or branched C₁-C₂₀-alkyl group, whereby the alkyl group can be interrupted by 1-6 oxygen atoms and/or substituted by 1-6 hydroxy groups, or radicals R² and R³ as well as R⁴ and R⁵ with the nitrogen atom to which they are bonded form a heterocyclic ring, which optionally can be substituted with 1-3 hydroxy groups.

Of the compounds of general formula I according to the invention, those are preferred in which L has the following meaning:

—CH₂CH₂—

—CH₂CH₂CH₂—

—CH₂—O—CH₂—

—CH₂CH(OH)CH₂—

—CH₂—O—CH₂CH₂—O—CH₂—

and R¹ has the following meaning:

—H

—CH₂COOH

—CH₂CH₂COOH

and X and Y are identical and mean the following groups:

—N[CH₂—CH(OH)—CH₂OH]₂

—NH—CH₂—CH(OH)—CH₂OH

—NH—CH(CH₂OH)₂

—N(CH₃)—CH₂—CH(OH)—CH(OH)—CH(OH)—CH(OH)—CH₂OH

—NH—CH₂—CH(OH)—CH(OH)—CH(OH)—CH(OH)—CH₂OH

and X and Y are not identical and X has the following meaning

—N[CH₂—CH(OH)—CH₂OH]₂

—NH—CH₂—CH(OH)—CH₂OH

—NH—CH(CH₂ OH)₂

and Y means the group —N(CH₃)—CH₂—CH(OH)—CH₂OH.

The compounds of general formula I with A and B in the meaning of a carbonyl group are obtained by reaction of compound II (J. of Org. Chem. Vol. 59, pages 1344-1350, 1994) with a dicarboxylic acid-monochloride of general formula III, whereby L has the above-indicated meaning.

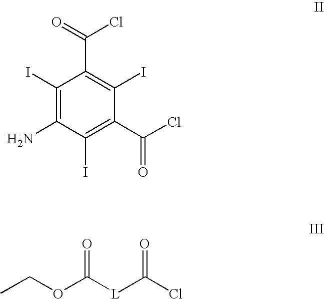

In this case, first obtained are compounds of general formula IV,

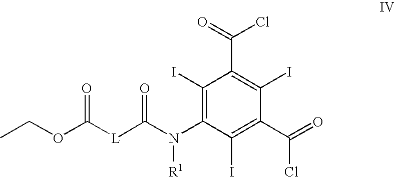

which are reacted with an amine of general formula V, whereby R² and R³ have the above-indicated meaning and free hydroxyl groups are optionally protected, to form compounds of general formula VI. As protective groups, acetyl groups or isopropylidine groups are suitable. This protective group technique is well-known to one skilled in the art.

HN—R²R³ v

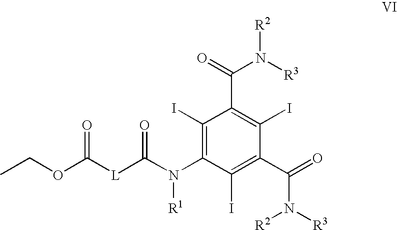

For the case that R¹=H, the ethyl ester is cleaved in VI by basic treatment, and the corresponding carboxylic acid is reacted with an amine of general formula VII to form products of general formula VIII

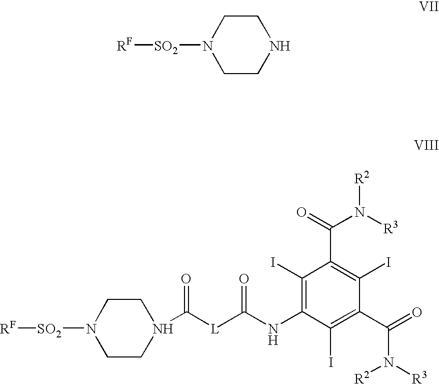

In this case, R^{F}, L, R² and R³ have the above-indicated meaning. The reaction is carried out according to the processes of acid activation that are known in general to one skilled in the art such as:

by reaction of the acid with dicyclohexylcarbodiimide, thionyl chloride, N-hydroxysuccinimide/dicyclohexylcarbodiimide, carbonyldiimidazole, 2-ethoxy-1-ethoxy-carbonyl-1,2-dihydroquinoline, oxalic acid dichloride or isobutyl chloroformate, in the way that is described in the literature:

Aktivierung von Carbonsäuren. [symbol] bersicht in Houben-Weyl, Methoden der Organischen Chemie [Activation of Carboxylic Acids. Survey in Houben-Weyl, Methods of Organic Chemistry], Volume XV/2, Georg Thieme Stuttgart, 19.

Aktivierung mit Carbodiimiden [Activation with Carbodiimides]. R. Schwyzer and H. Kappeler, Helv. 46: 1550 (1963).

E. Wünsch et al., Vol. 100: 173 (1967).

Aktivierung mit Carbodiimiden/Hydroxysuccinimid [Activation with Carbodiimides/Hydroxysuccinimide]: J. Am. Chem. Soc. 86: 1839 (1964) as well as J. Org. Chem. 53: 3583 (1988). Synthesis 453 (1972).

Anhydridmethode, 2-Ethoxy-1-ethoxycarbonyl-1,2-dihydrochinolin [Anhydride Methods, 2-Ethoxy-1-ethoxycarbonyl-1,2-dihydroquinoline]: B. Belleau et al., J. Am. Chem. Soc., 90: 1651 (1986), H. Kunz et al., Int. J. Pept. Prot. Res., 26: 493 (1985) and J. R. Voughn, Am. Soc. 73: 3547 (1951).

Imidazolid-Methode [Imidazolide Methods]: B. F. Gisin, R. B. Merrifield, D. C. Tosteon, Am. Soc. 91: 2691 (1969).

Säurechlorid-Methoden, Thionylchlorid [Acid Chloride Methods, Thionyl Chloride]: Helv., 42: 1653 (1959).

Oxalylchlorid [Oxalyl Chloride]: J. Org. Chem., 29: 843 (1964).

After cleavage of optionally present protective groups, the compounds of general formula I are obtained.

If, however, compounds with R¹ in the meaning of —CH₂COOH or —CH₂CH₂COOH are desired, the compounds of general formula VI are reacted with bromoacetic acid-tert-butyl ester or 2-bromopropionic acid-tert-butyl ester to form compounds of general formula IX, whereby R¹ stands for —CH₂CO₂-tert-butyl or —CH2CH2CO2-tert-butyl.

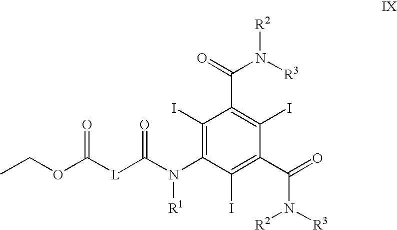

After the cleavage of the ethyl ester in IX, it is reacted with an amine of general formula VII to introduce a perfluorinated radical, and the protective groups in R¹, R², and R³ are ultimately removed.

It has been shown that the perfluoroalkyl-group-containing triiodoaromatic compounds of general formula I according to the invention are very well suited as contrast media for x-ray diagnosis, for ¹⁹F-NMR diagnosis and ¹⁹F-NMR spectroscopy and especially for the combined x-ray and NMR diagnosis. They are preferably usable as contrast media for visualizing the intravascular space (including vascular leakages), the liver and the bile duct, the gastrointestinal tract, the lymph tracts and atherosclerotic plaque. The compounds according to the invention can also be administered in combination with the contrast media based on heavy metal chelates (e.g., Magnevist®, Dotarem®, ProHance®). Moreover, the agents according to the invention can be used as ultrasonic contrast media.

Subjects of the invention are therefore also diagnostic agents that contain at least one of the compounds according to the invention and optionally physiologically compatible adjuvants and/or additives that are commonly found in galenicals for the formulation of pharmaceutical agents. The agents according to the invention are intended for enteral or parenteral administration. Suitable additives are, for example, physiologically harmless buffers (such as, e.g., tromethamine, bicarbonate, phosphate, citrate), stabilizers (such as, e.g., DTPA, sodium edetate, calcium-disodium edetate) or—if necessary—electrolytes (such as, e.g., sodium chloride) or—if necessary—antioxidants (such as, e.g., ascorbic acid) or else osmolality adaptation substances (such as, e.g., mannitol, glucose). If suspensions or solutions of the agents in water or physiological salt solution according to the invention are desired for enteral administration or other purposes, they are mixed with one or more adjuvants that are commonly used in galenicals (e.g., methyl cellulose, lactose, mannitol) and/or surfactants (e.g., lecithins, Tweens®, Myrj® and/or flavoring substances for taste correction (e.g., ethereal oils).

The concentration of the new x-ray contrast media in the aqueous medium depends completely on the x-ray diagnostic method. The common concentrations fluctuate in the ranges of 2 to 250 mmol/l, preferably 5 to 150 mmol/l. The iodine content of the solutions is usually in the range between 50 to 450 mg/ml, preferably 100 to 350 mg/ml.

The resulting agents are then optionally heat-sterilized. They are administered based on the iodine content and the x-ray diagnostic or question-statement method that is used generally at a dose of 5 mg of iodine/kg to 2000 mg of iodine/kg and in amounts of 1 to 1000 ml.

The-administration of the aqueous x-ray contrast medium solution can be carried out enterally or parenterally, thus orally, rectally, intravenously, intraarterially, intravascularly, intracutaneously, interstitially, subcutaneously (lymphography), or subarachnoidally (myelography).

The agents according to the invention show the high effectiveness that is necessary to load the body with the smallest possible amounts of foreign substances and the good compatibility that is necessary to maintain the non-invasive nature of the studies.

The compounds according to the invention are distinguished by a surprisingly low CMC (see Table I).

The CMC was determined by the concentration-dependent determination of the surface tension. The method is described by, i.a., Elworthy and Mysels; J. Colloid Interface Sci., 21, 331, 1966; Mukerjee, Mysels; The Critical Micelle Conc. Of Aqu. Surfactant Systems, 1971, US National Bureau of Standards and G. Brosowski, M. Rohr, G. Lehmann; Fette, Streifen, Anstrichsmittel [Fats, Soaps, Paints], 75, 335, 1973.

TABLE I
Critical Micelle Formation Concentration (CMC)

| Substance from the Example in the | CMC |
|---|---|
| Experimental Part | [mol/l] |
| 1 e | 6.4 × 10⁻⁵ |
| 2 c | 6.2 × 10⁻⁵ |
| 3 e | 5.4 × 10⁻⁵ |
| 4 c | 5.4 × 10⁻⁵ |
| 5 f | 1.2 × 10⁻⁴ |
| 6 e | 6.9 × 10⁻⁵ |

Experimental Part

Production of Bis-(2,2-dimethyl)-[1,3]dioxolan-4-yl-methyl)-amine:

152.21 g (1.10 mol) of potassium carbonate and 142.88 g (0.73 mol) of 4-bromomethyl-2,2-dimethyl-1,3-dioxolane were added to a solution of 243.16 g (1.10 mol) of 1-N-benzylamino-2,3-O-isopropylidine glycerol (production: Synthesis, 627, 1995) in 600 ml of acetonitrile, and it was refluxed for 10 hours. After filtration, it was concentrated by evaporation in a vacuum, and the crude product was purified by chromatography on a silica gel column (ethyl acetate/hexane 1:1+0.5% triethylamine). The yield of bis-(2,2-dimethyl)-[1,3]dioxolan-4-yl-methyl)-benzylamine was 179.98 g. TLC R_{f} 0.73 (CH₂Cl₂/MeOH 10:1).

¹H-NMR (CDCl₃): δ (ppm) 1.36 [s, 6H, O₂ (CH₃)₂], 1.44 [s, 6H, O₂(CH₃)₂], 2.74 [d, 4H, N(CH₂CHR)₂], 3.67 [dd, 2H, CHCH₂O], 3.83 [s, 2H, C_{benzyl}H₂], 4.04 [dd, 2H, CHCH₂O], 4.26 [m, 2H, CH(O)CH₂O], 7.29 [m, 5H, ArH]

MS (Cl): m/z (%) 336 (100) [M+H]⁺, 91 (4) [Benzyl]⁺

31.32 g (0.46 mol) of bis-(2,2-dimethyl-[1,3]dioxolan-4-yl-methyl)-benzylamine (see above) was dissolved in 400 ml of methanol, mixed with 3 g of Pd/carbon (10%) and stirred for 24 hours under a hydrogen atmosphere. After filtration, the solution was concentrated by evaporation in a vacuum. 18.2 g of bis-(2,2-dimethyl)-[1,3]dioxolan-4-yl-methyl)-amine was obtained as a viscous oil. TLC R_{f} 0.48 (CH₂ Cl₂/methanol 10:1).

¹H-NMR (CDCl₃): δ (ppm) 1.36 [s, 6H, O₂C(CH₃)₂], 1.47 [s, 6H, O₂C(CH₃)₂], 3.00 [m, 2H, NCH₂CH], 3.10 [m, 2H, NCH₂CH], 3.77 [dd, 1H, CHCH₂O], 3.83 [dd, 1H, CHCH₂O], 4.14 [dd, 2H, CHCH₂O], 4.59 [m, 2H, CH(O)CH₂O]

MS (CI): m/z (%) 246 (100) [M+H]⁺, 188 (6) [M-C₃H₆O]⁺

### EXAMPLE 1

1a)

({3,5-Bis-[bis-(2,2-dimethyl-[1,3]dioxolan-4-ylmethyl)-carbamoyl]-2,4,6-triiodophenylcarbamoyl}-methoxy-acetic Acid-ethyl Ester

22.1 g (0.09 mol) of bis-(2,2-dimethyl)-[1,3]dioxolan-4-yl-methyl)-amine and 16.6 ml (0.12 mol) of NEt₃ were introduced into 70 ml of DMF. A solution of 21.88 g (0.03 mol) of [(3,5-bis-chlorocarbonyl 2,4,6-triiodo-phenylcarbamoyl)-methoxy]-acetic acid-ethyl ester in 50 ml of DMF was now slowly added to the above at room temperature and stirred for 16 hours. It was first stirred for 14 hours at 40° C. and then again for 72 hours at room temperature; the solution was colored reddish-brown. After the filtration, the batch was completely concentrated by evaporation, and the crude product was purified by column chromatography (EtOAc 100%). 22.2 g (0.019 mol, 63.9%) of the title compound was obtained.

¹H-NMR (d₆-DMSO): δ (ppm) 1.31 [m, 27H, O₂C(CH₃)₂, OCH₂CH₃], 3.27 [m, 4H, NCH₂CH], 3.47 [m, 4H, NCH₂CH], 3.92 [m, 4H, CHCH₂O], 4.16 [m, 6H, (O)CCH₂OCH₂C(O), OCH₂CH₃], 4.33 [m, 6H CHCH₂O, CH(O)CH₂], 4.61 [m, 2H, CH(O)CH₂], 10.01 [br, 1H, ArNH]

MS (ESI): m/z (%) 1158(100) [M+H]⁺, 1100(3) [M-C₃H₆O]⁺

The starting material for this stage 1a—[(3,5-bis-chlorocarbonyl 2,4,6-triiodophenylcarbamoyl)-methoxy]-acetic acid-ethyl ester—was obtained in the following way:

24.75 g (125.9 mmol) of diglycolic acid-monoethyl ester in 50 ml of ethylene chloride was mixed with 9.2 ml of thionyl chloride and a drop of DMF, and it was refluxed for 15 minutes. The solution that was cooled to room temperature was added in drops to a suspension of 50 g (83.9 mmol) of 5-amino-2,4,6-triiodo-isophthalic acid-dichloride (see J. of Org. Chem. Vol. 59, pages 1344-1350, 1994) in 200 ml of ethylene chloride, and it was refluxed for 2 hours. The solution was concentrated by evaporation in a vacuum and chromatographed on a silica gel column. By elution with toluene/ethyl acetate, 54 g of [(3,5-bis-chlorocarbonyl 2,4,6-triiodo-phenylcarbamoyl)-methoxy]-acetic acid-ethyl ester was obtained as a light yellow powder.

1b)

[{3,5-Bis-[bis-(2,2-dimethyl-[1,3]dioxolan-4-ylmethyl)-carbamoyl]-2,4,6-triiodo-phenyl}-(2-ethoxycarbonylmethoxy-acetyl)-acetic Acid-Tert-Butyl Ester

14 g (0.012 mol) of the substance that is obtained according to Example 1a was dissolved in 70 ml of DMF, and 2.5 g (0.018 mol) of K₂CO₃ and 2.7 ml (0.018 mol) of bromoacetic acid-tert-butyl ester were added in succession. The batch was stirred for 16 hours at room temperature, it was then filtered, and the now clear, yellowish-colored solution was concentrated by evaporation in a rotary evaporator. After chromatographic purification (EtOAc 100%), 14.95 g of the title compound remains.

¹H-NMR (CDCl₃): δ (ppm) 1.39 [m, 36H, O₂C(CH₃)₂, OC(CH₃)₃, OCH₂CH₃], 3.48 [m, 10H, NCH₂CH, NCH₂C(O)], 4.11 [m, 14H, (O)CCH₂OCH₂C(O), OCH₂CH₃, CHCH₂O], 4.43 [m, 2H, CHCH₂O], 4.62 [m, 2H, CHCH₂O]

MS (ESI): m/z (%) 1272 (100) [M+H]⁺

1c)

[{3,5-Bis-[bis-(2,2-dimethyl-[1,3]dioxolan-4-ylmethyl)-carbamoyl]-2,4,6-triodo-phenyl}-tert-butoxycarbonylmethyl-carbanoyl)-methoxy]-acetic Acid

0.24 g (5.9 mmol) of NaOH and 5 g (3.93 mmol) of the substance that is produced according to Example 1b were dissolved in succession in 50 ml of ethanol, and 5 ml of water was added. It was stirred for 24 hours at room temperature and then neutralized with 2N HCl. The solution was concentrated by evaporation in a rotary evaporator, mixed with 100 ml of water and 200 ml of EtOAc and cooled in an ice bath. Then, it was acidified with 2N HCl to pH 2.8, and the phases were separated in a spherical separating funnel after 15 minutes of vigorous stirring. The aqueous phase was extracted twice with 50 ml of EtOAc, and the combined organic phases were dried on Na₂SO₄. Sodium sulfate was separated, and the clear solution was concentrated by evaporation in a rotary evaporator. After drying in an oil pump vacuum, 3.78 g (3.04 mmol, 77.1%) of the title compound was obtained. TLC R_{f} 0.48 (EtOAc/MeOH 1:1).

MS (ESI): m/z (%) 1244 (68) [M+H]⁺, 1188 (24) [M-C₃H₆O]⁺, 1118 (4) [M-I]⁺

1d)

[{3,5-Bis-[bis-(2,2-dimethyl-[1,3]dioxolan-4-ylmethyl)-carbamoyl]-2,4,6-triiodo-phenyl}-(2-{2-[4-(heptadecafluorooctane-1-sulfonyl)piperazin-1-yl]-2-oxo-ethoxy}-acetyl)-amino]-acetic Acid-Tert-Butyl Ester

3.78 g (3.04 mmol) of the substance that is obtained according to Example 1c was dissolved in 30 ml of DMF. 0.35 g (3.04 mmol) of N-hydroxysuccinimide and 0.63 g (3.04 mmol) of DCC were now added in succession; the batch was stirred for 16 hours at room temperature.

The NHS-active ester was mixed with 1.73 g (3.04 mmol) of 1-(heptadecafluorooctane-1-sulfonyl)-piperazine (production: WO 97/26017 Example 27a) and 1.03 ml (6.04 mmol) of DIPEA and stirred for 24 hours at room temperature. The precipitated dicyclohexylurea was separated on a G4 frit, and the solvent was removed in a vacuum. 6.93 g (100%) of the crude product of the title compound remains.

H-NMR (CDCl₃): δ (ppm) 1.39 [m, 33H, O₂C(CH₃)₂, OC(CH₃)₃], 3.48 [m, 18H, NCH₂ CH, piperazine-CH₂, NCH₂ C(O)], 4.1 [m, 8H, CHCH₂O, (O)CCH₂OCH₂C(O)], 4.41 [m, 4H, CHCH₂O], 4.61 [m, 4H, CHCH₂O]

MS (ESI): m/z (%) 1794 (18) [M+H]⁺, 1738 (7) [M-C₃H₆O]⁺

1e)

[{3,5-Bis-[bis-(2,3-dihydroxy-propyl)-carbamoyl]-2,4,6-triiodo-phenyl}-(2-{2-[4-(heptadecafluorooctane-1-sulfonyl)piperazin-1-yl]-2-oxo-ethoxy}-acetyl)-amino]-acetic Acid

10 ml of trifluoroacetic acid was added at 0° C. to 30 ml of a solution that consists of 6.93 g (3.82 mmol) of the substance, obtained according to Example 1d, in dichloromethane, and it was stirred first for 14 hours under cold conditions and later at room temperature. Then, the solution was completely concentrated by evaporation, the product was dissolved in 20 ml of methanol, and it was brought to pH 1 after the addition of 50 ml of water with 2N HCl. The batch was now stirred for 16 hours at room temperature, and in this case a fine dicyclohexylurea precipitate settled out. The precipitate was removed by filtration, then the methanol was removed for the most part in a rotary evaporator, and the aqueous solution was membrane-filtered (nitrocellulose, Satorius Company, 0.2 μm). After the neutralization with 2N NaOH, the product was purified by ultrafiltration (regenerated cellulose, Amicon Company, Filter Code YM3, NMWL 3000, Lot No. KOMN K696). After the retentate is freeze-dried, 2.9 g (1.84 mmol, 48%) of the title compound remains as a colorless solid substance.

MS (ESI): m/z (%) 1600 (9) [M+Na]⁺, 1578 (71) [M+H]⁺, 1452 (5 [M-I]⁺, 1326 (4) [M-2I]⁺

### EXAMPLE 2

2a)

({3,5-Bis-[bis-(2,2-dimethyl-[1,3]dioxolan-4-ylmethyl)-carbamoyl]-2,4,6-triiodo-phenylcarbamoyl}-methoxy-acetic Acid

10.91 g (9.43 mmol) of ({3,5-bis-[bis-(2,2-dimethyl-[1,3]dioxolan-4-ylmethyl)-carbamoyl]-2,4,6-triiodo-phenylcarbamoyl}-methoxy-acetic acid-ethyl ester (see Example 1a) was dissolved in 50 ml of methanol, added with 14.2 ml (28.4 mmol) of 2N NaOH and stirred at room temperature for 14 hours. The course of the reaction was monitored by TLC. It was neutralized with 2N HCl, and then methanol was removed in a rotary evaporator. The aqueous solution was then mixed with 50 ml of EtOAc, cooled in an ice bath and acidified with dilute HCl to pH 2.8. It was stirred for 30 minutes under cold conditions, the emulsion was transferred into a spherical separating funnel, and the organic phase was separated. The acidic aqueous phase was extracted three more times with 20 ml of EtOAc, then the combined organic phases were dried on Na₂ SO₄. EtOAc was removed in a rotary evaporator, and after drying, 9.46 g (8.38 mmol, 89%) of the title compound was obtained as a yellow solid. TLC R_{f} 0.72 (EtOAc/MeOH 4:1).

¹H-NMR (d₆-DMSO): δ (ppm) 1.36 [m, 24H, O₂C(CH₃)₂], 3.35 [m, 8H, NCH₂CH], 3.88 [m, 4H, CHCH₂O], 4.11 [m, 4H, CHCH₂O], 4.2 [s, 2H, (O)CCH₂OCH₂C(O)], 4.26 [s, 2H, (O)CCH₂OCH₂C(O)], 4.32 [m, 2H, CHCH₂O], 4.44 [m, 2H, CHCH₂O], 10.04 [m, 1H, ArNH], 12.82 [s, 1H, COOH]

MS (ESI): m/z (%) 1130 (100) [M+H]⁺, 1072 (18) [M-C₃H₆O]⁺

2b)

N,N,N′,N′-Tetrakis-(2,2-dimethyl-[1,3]dioxolan-4-ylmethyl)-5-(2-{2-[4-(heptadecafluorooctane-1-sulfonyl)-piperazin-1-yl]-2-oxo-ethoxy}-acetylamino)-2,4,6-triiodoisophthalamide

3.01 g (2.66 mmol) of ({3,5-bis-[bis-(2,2-dimethyl-[1,3]dioxolan-4-ylmethyl)-carbamoyl]-2,4,6-triiodo-phenylcarbamoyl}-methoxy-acetic acid (Example 2a) and 1.67 g (2.93 mmol) of 1-(heptadecafluorooctane-1-sulfonyl)-piperazine (production: WO 97/26017 Example 27a) were dissolved in succession in 50 ml of THF. It was now cooled to 0° C. in an ice bath, and then 0.549 g (2.66 mmol) of DCC was introduced with a spatula. The batch was stirred for another 4 hours in an ice bath, then for 14 hours at room temperature. As early as 30 minutes after DCC addition, a cloudiness of the solution that was produced by the dicyclohexylurea that was forming could be detected. The suspension was membrane-filtered (Millipore Company, PTFE 0.2 μm), and THF was removed in a rotary evaporator. The crude product was purified by chromatography (EtOAc 100%). 4.14 g (2.46 mmol, 92.7%) of the title compound was obtained as a pale yellow solid. TLC R_{f} 0.18 (EtOAc).

¹H-NMR (CDCl₃): δ (ppm) 1.40 [m, 24H, O₂C(CH₃)₂], 3.5 [m, 16H, NCH₂CH, piperazine-CH₂], 4.1 [m, 6H, CHCH₂O, (O)CCH₂OCH₂C(O)], 4.31 [m, 4H, (O)CCH₂OCH₂C(O), (O)CHCH₂O], 4.46 [m, 4H, CHCH₂O, CHCH₂O], 4.62 [m, 2H, CHCH₂O], 9.85 [m, 1H, ArNH]

MS (ESI): m/z (%) 1680 (70) [M+H]⁺, 1622 (5) [M-C₃H₆O]⁺

2c)

N,N,N′,N′-Tetrakis-(2,3-dihydroxy-propyl)-5-(2-{2-[4-(heptadecafluorooctane-1-sulfonyl-piperazin-1-yl]-2-oxo-ethoxy}-acetylamino)-2,4,6-triiodo-isophthalamide

4.14 g (2.46 mmol) of N,N,N′,N′-tetrakis-(2,2-dimethyl-[1,3]dioxolan-4-ylmethyl)-5-(2-{2-[4-(heptadecafluorooctane-1-sulfonyl)-piperazin-1-yl]-2-oxo-ethoxy}-acetylamino)-2,4,6-triiodo-isophthalamide (see Example 2b) was dissolved in 25 ml of methanol, and 10 ml of 2N HCl as well as 50 ml of bidistilled water were added in succession and stirred at room temperature for 20 hours. After neutralization with 2N NaOH, the light yellow solution was membrane-filtered (nitrocellulose, Sartorius Company, 0.1 μm) to remove dicyclohexylurea residue, then further purification was carried out by ultrafiltration (regenerated cellulose, Amicon Company, Filter Code YM3, NMWL 3000, Lot No. KOMN K696). The retentate was freeze-dried, and 2.88 g (1.9 mmol, 77%) of the title compound remains in the form of a colorless, powdery solid.

MS (ESI): m/z 1519 [M+H]+

C_{Cld.}: 28.5 H_{Cld.}: 2.70 N_{Cld.}: 4.6 I_{Cld.}: 25.1 F_{Cld.}: 21.3 C_{Fnd.}: 27.46 H_{Fnd.}: 3.04 N_{Fnd.}: 4.27 I_{Fnd.}: 24.39 F_{Fnd.}: 21.27

H₂O(KF): 3.45%

### EXAMPLE 3

3a)

N-{3,5-Bis-[bis-(2,2-dimethyl-[1,3]dioxolan-4-ylmethyl)-carbamoyl]-2,4,6-triiodophenyl}-succinic Acid-Ethyl Ester

17.91 g (0.073 mol) of bis-(2,2-dimethyl)-[1,3]dioxolan-4-yl-methyl)-amine, 21.15 g (0.029 mol) of N-(3,5-bis-chlorocarbonyl-2,4,6-triiodo-phenyl)-succinic acid-ethyl ester and 10.5 ml (0.076 mol) of NEt₃ were dissolved in 250 ml of DMF and stirred for 2 hours at 45° C. and then for 20 hours at room temperature. The precipitated hydrochloride was separated by filtration, and the now clear brown solution was concentrated by evaporation in a vacuum. The reddish-brown oily residue was dissolved in 250 ml of EtOAc and extracted four times with 50 ml of 0.1 M NaHSO₄ solution. The organic phase was ultimately washed twice with 30 ml of a saturated NaCl solution and dried on Na₂SO₄. After concentration by evaporation, the purification of the crude product was carried out by column chromatography (EtOAc/hexane 3:1). 17.64 g (0.015 mol; 53.3%) of the title compound remains as a pale yellow solid. TLC R_{f} 0.15 (EtOAc/hexane 3:1).

¹H-NMR (d₆-DMSO): δ (ppm) 1.19 [t, ³J=7.5 Hz, 3H, OCH₂CH₃], 1.35 [d, 24H, O₂C(CH₃)₂], 2.6 [s, 4H, (O)CCH₂CH₂C(O)], 3.5 [m, 4H, NCH₂CH], 3.8 [m, 6H, NCH₂CH, CHCH₂O], 4.12 [m, 4H, CHCH₂O, OCH₂CH₃], 4.4 [m, 4H, CHCH₂O], 10.09 [m, 1H, ArNH]

MS (ESI): m/z (%) 1142 (100) [M+H]⁺

The starting material for this stage 3a —N-(3,5-bis-chlorocarbonyl-2,4,6-triiodo-phenyl)-succinic acid-ethyl ester—was obtained in the following way:

20.0 g of succinic acid-monoethyl ester in 50 ml of ethylene chloride was mixed with 9.2 ml of thionyl chloride and one drop of DMF and refluxed for 15 minutes. The solution that was cooled to room temperature was added in drops to a suspension of 50 g (83.9 mmol) of 5-amino-2,4,6-triiodo-isophthalic acid-dichloride (see J. of Org. Chem. Vol. 59, pages 1344-1350, 1994) in 200 ml of ethylene chloride, and it was refluxed for 2 hours. The solution was concentrated by evaporation in a vacuum and chromatographed on a silica gel column. By elution with toluene/ethyl acetate, 41.5 g of N-(3,5-bis-chlorocarbonyl-2,4,6-triiodo-phenyl)-succinic acid-ethyl ester was obtained as a light yellow powder.

3b)

N-{3,5-Bis-[bis-(2,2-dimethyl-[1,3]dioxolan-4-ylmethyl)-carbamoyl]-2,4,6-triiodophenyl}-N-tert-butoxycarbonylmethyl-succinic Acid-Ethyl Ester

A solution of 9.8 g (8.6 mmol) of N-{3,5-bis-[bis-(2,2-dimethyl-[1,3]dioxolan-4-ylmethyl)-carbamoyl]-2,4,6-triiodo-phenyl}-succinic acid-ethyl ester (see Example 3a) in 20 ml of DMF was mixed with 1.8 g (12.9 mmol) of K₂ CO₃ and 1.8 ml (12.9 mmol) of bromoacetic acid-tert-butyl ester and stirred for 10 hours at 60° C. Then, it was filtered, and the solvent was removed in a vacuum. The crude product was purified by chromatography (EtOAc/EtOH 19:1). The yield was 9.8 g (7.80 mmol, 90.7%) of the title compound. TLC R_{f} 0.85 (EtOAc).

¹H-NMR (CDCl₃): δ (ppm) 1.24 [m, 3H, OCH₂CH₃], 1.42 [m, 33H, O₂C(CH₃)₂, OC(CH₃)₃], 2.35 [m, 2H, (O)CCH₂CH₂C(O)], 2.63 [m, 2H, (O)CCH₂CH₂C(O)], 3.45 [m, 8H, NCH₂CH], 3.81 [m, 2H, NCH₂C(O)], 4.09 [m, 10H, CHCH₂O, OCH₂CH₃], 4.48 [m, 4H, CHCH₂O]

MS (FAB): m/z (%) 1256 (4) [M+H]⁺, 1198 (100) [M-C₄H₉]⁺, 1072 (18) [M-C₄H₉—I]⁺

3c)

N-{3,5-Bis-[bis-(2,2-dimethyl-[1,3]dioxolan-4-ylmethyl)-carbamoyl]-2,4,6-triiodophenyl}-N-tert-butoxycarbonylmethyl-succinic Acid

9.8 g (7.8 mmol) of N-{3,5-bis-[bis-(2,2-dimethyl-[1,3]dioxolan-4-ylmethyl)-carbamoyl]-2,4,6-triiodo-phenyl}-tert-butoxycarbonylmethyl-succinic acid-ethyl ester (see Example 3b) was dissolved in 20 ml of ethanol, 0.936 g (23.4 mmol) of NaOH was added to it, and it was stirred for 12 hours at 60° C. Then, it was completely concentrated by evaporation, the residue was suspended in 50 ml of EtOAc/50 ml of water, and it was acidified to pH 2.8 in an ice bath with 0.1 N HCl. The phases were separated, the aqueous phase was extracted several more times with a little EtOAc, and the combined organic phases were dried on Na₂SO₄. The dessicant was separated, and the clear solution was concentrated by evaporation in a vacuum. The crude product was purified by chromatography (EtOAc/EtOH 5:1). 6.3 g (5.13 mmol, 52%) of the title compound was obtained. TLC R_{f} 0.78 (EtOAc/MeOH).

¹H-NMR (d₆-DMSO): δ (ppm) 1.36 [m, 33H, O₂C(CH₃)₂, OC(CH₃)₃], 2.25 [m, 2H, (O)CCH₂CH₂C(O)], 2.35 [m, 2H, (O)CCH₂CH₂C(O)], 3.52 [m, 8H, NCH₂CH], 3.83 [m, 2H, NCH₂C(O)], 4.05 [m, 8H, CHCH₂O], 4.36 [m, 4H, CHCH₂O]

MS (ESI): m/z (%) 1228 (87) [M+,H]⁺, 1172 (68) [M-C₄H₉]⁺, 1046 [M-C₄H₉—I]⁺

3d)

[{3,5-Bis-[bis-(2,2-dimethyl-[1,3]dioxolan-4-ylmethyl)-carbamoyl]-2,4,6-triiodo-phenyl}-{4[4-(heptadecafluorooctane-1-sulfonyl)piperazin-1-yl]-4-oxo-butyryl}-amino)-acetic Acid-Tert-Butyl Ester

In 75 ml, 5.1 g (4.15 mmol) of N-{3,5-bis-[bis-(2,2-dimethyl-[1,3]dioxolan-4-ylmethyl)-carbamoyl]-2,4,6-triiodo-phenyl}-N-tert-butoxycarbonylmethyl-succinic acid (see Example 3c) and 0.478 g (4.15 mmol) of NHS were dissolved in succession. After the addition of 0.856 g (4.15 mmol) of DCC, the batch was stirred for 24 hours at room temperature. The active ester was now further reacted by adding 2.36 g (4.15 mmol) of 1-(heptadecafluorooctane-1-sulfonyl)-piperazine (production: WO 97/26017 Example 27a) and 0.85 ml (4.97 mmol) of DIPEA. After 24 hours, the solvent was removed in a vacuum, the residue was taken up in a little methanol and purified by chromatography (dichloromethane/methanol 19:1). The yield was 6.15 g (3.46 mmol, 83%) of the title compound. TLC R_{f} 0.77 (MeCl₂/MeOH 9:1)

¹H-NMR (CDCl₃): δ (ppm) 1.41 [m, 33H, O₂C(CH₃)₂, OC(CH₃)₃], 2.35 [m, 2H, (O)CCH₂CH₂C(O)], 2.74 [m, 2H, (O)CCH₂CH₂C(O)], 3.5 [m, 18H, NCH₂CH, piperazine CH₂, NCH₂C(O)], 4.1 [m, 8H, CHCH₂O], 4.42 [m, 2H, CHCH₂O], 4.63 [m, 2H, CHCH₂O]

MS (ESI): m/z (%) 1778 (25) [M+H]⁺

3e)

[{3,5-Bis-[bis-(2,3-dihydroxy-propyl)-carbamoyl]-2,4,6-triiodo-phenyl}-{4[4-(heptadecafluorooctane-1-sulfonyl)-piperazin-1-yl]-4-oxo-butyryl}-amino)-acetic Acid

For hydrolytic cleavage of the protective groups, 6.15 g (3.46 mmol) of [{3,5-bis-[bis-(2,2-dimethyl-[1,3]dioxolan-4-ylmethyl)-carbamoyl]-2,4,6-triiodo-phenyl}-{4[4-(heptadecafluorooctane-1-sulfonyl)piperazin-1-yl]-4-oxo-butyryl}-amino)-acetic acid-tert-butyl ester (see Example 3d) was dissolved in 50 ml of dichloromethane, mixed with 25 ml of TFA and stirred for 24 hours at room temperature. The dichloromethane/TFA mixture was removed in a rotary evaporator, the residue was treated twice in each case with 60 ml of toluene and again concentrated by evaporation. After drying, a 1H-NMR spectrum was prepared, since, however, signals could still be detected that could be assigned to the acetonide protective groups; the crude product was dissolved again in 50 ml of methanol and brought to pH 1.7 with 0.1 M HCl. After 48 hours, it was neutralized and then completely concentrated by evaporation, and the flask contents foamed vigorously. The residue was taken up again in 200 ml of water and membrane-filtered (nitrocellulose (Millipore Company), pore size 0.1 μm). The final purification was carried out by ultrafiltration to a conductivity <5 μS in an AMICON stirred cell (regenerated cellulose, Filter Code YM3, NMWL 3000, Lot No. KODN K003).

The retentate was freeze-dried and characterized. The product was purified by a preparative HPLC. 3.24 g (2.07 mmol, 60%) of the title compound remains.

MS (ESI): m/z (%) 1562 (100) [M+H]⁺, 1436 (5) [M-I]⁺

C_{Cld.}: 29.2 H_{Cld.}: 2.7 N_{Cld.}: 4.5 I_{Cld.}: 24.4 F_{Cld.}: 20.7 C_{Fnd.}: 28.61 H_{Fnd.}: 3.14 N_{Fnd.}: 4.43 I_{Fnd.}: 22.99 F_{Fnd.}: 20.56

H₂O(KF): 2.82%

### EXAMPLE 4

4a)

N-{3,5-Bis-[bis-(2,2-dimethyl-[1,3]dioxolan-4-ylmethyl)-carbamoyl]-2,4,6-triiodo-phenyl}-succinic Acid

17.72 g (0.0155 mol) of N-{3,5-bis-[bis-(2,2-dimethyl-[1,3]dioxolan-4-ylmethyl)-carbamoyl]-2,4,6-triiodo-phenyl}-succinic acid-ethyl ester was dissolved in 50 ml of methanol and mixed with 23.28 ml (0.047 mol) of 2N NaOH. The batch was stirred for 72 hours at room temperature, then it was neutralized with 2N HCl and then methanol was removed in a rotary evaporator. The aqueous solution was then mixed with 200 ml of EtOAc, cooled in an ice bath and slowly brought to pH 2.8 with 2N HCl. The emulsion was stirred for 15 minutes, transferred into a spherical separating funnel, and the organic phase was separated. The aqueous phase was extracted three more times with 50 ml of EtOAc each, the organic phases were combined and dried on Na₂SO₄. The desiccant was removed, and the solution was concentrated by evaporation in a vacuum. After drying, 17.77 g (0.0155 mol, 100%) of the title compound was obtained. TLC R_{f} 0.08 (EtOAc)

¹H-NMR (CD₃OD): δ (ppm) 1.49 [m, 24H, O₂C(CH₃)₂], 2.74 [m, 4H, (O)CCH₂CH₂C(O)], 3.53 [m, 8H, NCH₂CH], 3.98 [m, 8H, CHCH₂O], 4.47 [m, 2H, CHCH₂O], 4.63 [m, 2H, CHCH₂O]

MS (ESI): m/z (%) 1114 (66) [M+H]⁺, 1056 (33) [M-C₃H₆O]⁺

4b)

N,N,N′,N′-Tetrakis-(2,2-dimethyl-[1,3]dioxolan-4-ylmethyl)-5-{4-[4-(heptadecafluorooctane-1-sulfonyl)-piperazin-1-yl]-4-oxo-butyrylamino}-2,4,6-triiodo-isophthalamide

4.16 g (3.74 mmol) of N-{3,5-bis-[bis-(2,2-dimethyl-[1,3]dioxolan-4-ylmethyl)-carbamoyl]-2,4,6-triiodo-phenyl}-succinic acid and 2.34 g (4.11 mmol) of 1-(heptadecafluorooctane-1-sulfonyl)-piperazine (production: WO 97/26107 Example 27a) were dissolved in THF. The solution was then cooled in an ice bath to 0° C. and mixed with 0.772 g (3.74 mmol) of DCC. It was stirred for 3 hours under cold conditions and then for 16 hours at room temperature; after 2 hours, the solution became cloudy from the precipitating dicyclohexylurea.

The urea was separated on a G4 frit, and THF was then removed in a vacuum. The crude product was purified by chromatography (EtOAc/hexane 4:1). 3.05 g (1.83 mmol, 49%) of the title compound was obtained. TLC R_{f} 0.16 (EtOAc).

¹H-NMR (CD₃OD): δ (ppm) 1.38 [m, 24H, O₂C(CH₃)₂], 2.87 [m, 4H, (O)CCH₂CH₂C(O)], 3.6 [m, 16H, NCH₂CH, piperazine CH₂], 4.05 [m, 8H, CHCH₂O], 4.48 [m, 2H, CHCH₂], 4.64 [m, 2H, CHCH₂]

MS (ESI): m/z (%) 1664 (13) [M+H]⁺

4c)

N,N,N′,N′-Tetrakis-(2,3-dihydroxy-propyl)-5-{4-[4-(heptadecafluorooctane-1-sulfonyl)-piperazin-1-yl]-4-oxo-butyrylamino}-2,4,6-triiodo-isophthalamide

At room temperature, 3.05 g (1.83 mmol) of N,N,N′,N′-tetrakis-(2,2-dimethyl-[1,3]dioxolan-4-ylmetnyl)-5-{4-[4-(heptadecafluorooctane-1-sulfonyl)-piperazin-1-yl]-4-oxo-butyrylamino}-2,4,6-triiodo-isophthalamide was dissolved in 25 ml of methanol, 80 ml of water was added, and it was acidified to pH 1.8 with 2N HCl. The batch was stirred for 48 hours, then it was neutralized with 2N NaOH and then membrane-filtered (Sartorius Company, nitrocellulose, 0.1 μm). Methanol residue was removed for the most part in a rotary evaporator, and the aqueous solution was ultrafiltered after being diluted to a volume of 350 ml (Amicon stirred cell, regenerated cellulose, Filter Code YM3, 3000 NMWL, Lot No. KOMN K696, Amicon Company). The retentate was freeze-dried, and 1.51 g (1.00 mmol, 55%) of the title compound remains.

MS (ESI): m/z 1504 [M+H]⁺

### EXAMPLE 5

5a)

5-Amino-2,4,6-triiodo-N,N′-dimethyl-N,N′-bis-(2,3,4,5,6-pentahydroxy-hexyl)-isophthalamide

14.9 g (76.3 mmol) of N-methylglucamine was suspended in 50 ml of DMF and mixed with 11 ml (79.4 mmol) of NEt₃. At room temperature, 120 ml of a solution of 20 g (33.6 mmol) of 5-amino-2,4,6-triiodo-isophthaloyl dichloride (see J. of Org. Chem. Vol. 59, pages 1344-1350, 1994) in DMF was slowly added in drops to this suspension and stirred for 72 hours. Since in TLC, no starting material was visible any longer, the solution was filtered to separate the precipitated hydrochloride, and the now clear reddish-brown solution was concentrated by evaporation in a vacuum. The crude product was acetylated without further purification (see 5b).

MS (FAB): n/z (%) 914 (13) [M+H]⁺

5b)

Acetic Acid-2,3,4-triacetoxy-1-[1-acetoxy-2-({3-amino-2,4,6-triiodo-5-[methyl-(2,3,4,5,6-pentaacetoxy-hexyl)-carbamoyl]-benzoyl}-methyl-amino)ethyl]-butyl Ester

The crude product from Example 5a (33.6 mmol) was refluxed together with 80 ml (0.85 mol) of acetic acid anhydride and 70 ml (0.87 mol) of pyridine for the period of 3.5 hours, then it was stirred for another 16 hours at room temperature. The dark red solution was poured into an ice bath to form 600 ml of 10% hydrochloric acid and then another 300 ml of EtOAc was added. After 30 minutes of vigorous stirring, it was transferred into a spherical separating funnel, and the organic phase was separated; the aqueous phase was extracted three more times with 100 ml of EtOAc in each case. The combined organic phases were finally extracted twice more with 50 ml of 0.1N hydrochloric acid and dried on Na₂SO₄.

The crude product was purified by column chromatography (EtOAc/hexane 3:1). TLC R_{f} 0.11 (EtOAc/hexane 3:1). The yield was 40.8 g (30.6 mmol, 91%) of the title compound.

¹H-NMR (CDCl₃): δ (ppm) 2.12 [m, 30H, O(O)CCH₃], 2.86 [dd, 6H, N(CH₃)CH₂], 3.77 [m, 4H, N(CH₃)CH₂], 4.13 [m, 2H, CH₂OAc], 4.27 [m, 2H, CH₂OAc], 5.05 [s, 2H, ArNH₂], 5.16 [m, 2H CHOAc], 5.38 [m, 2H CHOAc], 5.53 [m, 2H CHOAc], 5.68 [m, 2H, CHOAc]

MS (FAB): m/z (%) 1333 (100) [M+H]⁺, 1273 (45) [M-C₂H₄O₂]⁺, 1207 (60) [M-I]⁺, 1082 (22) [M-21]⁺

5c)

({2,4,6-Triiodo-3,5-bis-[methyl-(2,3,4,5,6-pentaacetoxy-hexyl)-carbamoyl]-phenylcarbamoyl}-methoxy)-acetic Acid-Tert-Butyl Ester

4.28 g (0.0225 mol) of diglycolic acid-mono-tert-butyl ester was dissolved in 8 ml of dichloromethane and 60 ml of DMA, and the solution was cooled in an ice bath to 0° C. After 1.63 ml (0.025 mol) of thionyl chloride was added slowly, it was stirred for 1.5 hours in an ice bath, and then 15 g (0.0112 mol) of the substance of Example 5b —acetic acid-2,3,4-triacetoxy-1-[1-acetoxy-2-({3-amino-2,4,6-triiodo-5-[methyl-(2,3,4,5,6-pentaacetoxy-hexyl)-carbamoyl]-benzoyl}-methyl-amino)ethyl]-butyl ester—was added to it in portions. After three hours of stirring in an ice bath, it was stirred at room temperature for another 72 hours. The color of the solution changed from pale yellow to reddish-brown. DMA was removed in a vacuum, the brownish residue was taken up in 100 ml of EtOAc and extracted three times with 20 ml of 1 M NaHCO₃ solution. The organic phase was dried on Na₂SO₄, concentrated by evaporation and purified by chromatography (EtOAc/hexane 3:1). 6.97 g (4.63 mmol, 41.3%) of the title compound was obtained in the form of a light yellow solid substance. TLC R_{f} 0.14 (EtOAc/hexane 4:1).

¹H-NMR (CDCl₃): δ (ppm) 1.50 [s, 9H, OC(CH₃)₃], 2.13 [m, 30H, O(O)CCH₃], 2.90 [m, 6H, N(CH₃)CH₂], 3.76 [m, 4H, N(CH₃)CH₂], 4.12 [m, 4H, CH₂OAc, (O)CCH₂OCH₂C(O)], 4.26 [m, 4H, CH₂OAc, (O)CCH₂OCH₂C(O)], 5.15 [m, 2H, CHOAc], 5.37 [m, 2H, CHOAc], 5.52 [m, 2H, CHOAC], 5.71 [m, 2H, CHOAc], 9.10 [d, 1H, ArNHC(O)]

MS (ESI): m/z (%) 1506 (100) [M+H]⁺, 1450 (52) [M-C₄H₉]⁺

5d)

({2,4,6-Triiodo-3,5-bis-[methyl-(2,3,4,5,6-pentaacetoxy-hexyl)-carbamoyl]-phenylcarbamoyl}-methoxy)-acetic Acid

A solution that consists of 3.35 g (2.22 mmol) of ({2,4,6-triiodo-3,5-bis-[methyl-(2,3,4,5,6-pentaacetoxy-hexyl)-carbamoyl]-phenylcarbamoyl}-methoxy)-acetic acid-tert-butyl ester (see Example 5c) in 40 ml of dichloromethane was mixed at room temperature with 20 ml of trifluoroacetic acid and stirred for 24 hours. It was completely concentrated by evaporation, and the residue was mixed twice with 50 ml of toluene to remove the last TFA residue and again evaporated to the dry state. The product was dried in an oil pump vacuum, and 3.22 g (2.22 mmol, 100%) of the title compound remains. TLC R_{f} 0.09 (EtOAc 100%).

¹H-NMR (CDCl₃): δ (ppm) 2.11 [m, 30H, O(O)CCH₃], 2.92 [m, 6H, N(CH₃)CH₂], 3.83 [m, 4H, N(CH₃)CH₂], 4.13 [m, 2H, (O)CCH₂OCH₂C(O)], 4.28 [m, 6H, CH₂OAc, (O)CCH₂OCH₂C(O)], 5.15 [m, 2H, CHOAc], 5.38 [m, 2H, CHOAc], 5.51 [m, 2H, CHOAc], 5.67 [m, 2H, CHOAc], 8.96 [d, 1H, ArNHC(O)], 9.84 [br, 1H, COOH]

MS (ESI): m/z (%) 1450 (100) [M+H]⁺, 1390 (4) [M-C₂H₄O₂]⁺

5e)

Acetic Acid-2,3,4-triacetoxy-1-{1-acetoxy-2-[methyl-(2,4,6-triiodo-3-[methyl(2,3,4,5,6pentaacetoxy-hexyl)-carbamoyl]-5-{2-[2-oxo-2-(4-pentafluoroethanesulfonyl-piperazin-1-yl)-ethoxy]-acetylamino}-benzoyl)-amino]-ethyl}-butyl Ester

In 50 ml of THF, 3.22 g (2.22 mmol) of ({2,4,6-triiodo-3,5-bis-[methyl-(2,3,4,5,6-pentaacetoxy-hexyl)-carbamoyl]-phenylcarbamoyl}-methoxy)-acetic acid and 1.63 g (2.87 mmol) of 1-(heptadecafluorooctane-1-sulfonyl)-piperazine (production: WO 97/26017 Example 27a) were dissolved in succession, and the solution was then cooled in an ice bath. 0.493 g (2.39 mmol) of DCC was added under cold conditions, and it was stirred for 72 hours after warming to room temperature. The solvent was then removed in a rotary evaporator, the crude product was taken up in a little EtOAc and purified by column chromatography (EtOAc/hexane 3:1 to EtOAc 100%). The yield was 2.68 g (1.34 mmol, 60.4%) of the title compound. TLC R_{f} 0.21 (EtOAc 100%).

¹H-NMR (CD₃OD): δ (ppm) 2.11 [m, 30H, O(O)CCH₃], 2.94 [d, 6H, N(CH₃)CH₂], 3.49 [m, 4H, piperazine CH₂], 3.79 [m, 8H, N(CH₃)CH₂, piperazine CH₂], 4.11 [m, 2H, CH₂OAc], 4.32 [m, 4H, CH₂OAc, (O)CCH₂OCH₂C(O)], 4.55 [s, 2H, (O)CCH₂OCH₂C(O)], 5.16 [m, 2H, CHOAc], 5.45 [m, 2H, CHOAc], 5.55 [m, 2H, CHOAc], 5.70 [m, 2H, CHOAc].

MS (ESI): m/z (%) 2000 (100) [M+H]⁺

5f)

2,4,6-Triiodo-N,N′-dimethyl-5-{2-[2-oxo-2-(4-(heptadecafluorooctane-1-sulfonyl)-piperazin-1-yl]-ethoxy]-acetylamino}-N,N′-bis-(2,3,4,5,6-pentahydroxyhexyl)-isophthalamide

2.68 g (1.34 mmol) of the compound that is produced according to Example 5e was dissolved in 40 ml of methanol and mixed at room temperature with 8.5 ml (17.0 mmol) of 2N NaOH solution. The batch was stirred for 48 hours at room temperature, then it was diluted with water to 200 ml, and the suspension was membrane-filtered (Millipore Company, regenerated cellulose, 0.45 μm). The solution was then ultrafiltered to a conductivity <10 μS, and the retentate was freeze-dried (Amicon Company, regenerated cellulose, Filter Code YM3, 3000 NMWL, Lot No. KOCNK709). The yield was 1.132 g (0.717 mmol) of the title compound, which was obtained as a colorless solid substance.

MS (ESI): m/z (%) 1580 (50) [M+H]⁺

C_{Cld.}: 28.90 H_{Cld.}: 2.87 N_{Cld.}: 4.43 I_{Cld.}: 24.10 C_{Fnd.}: 27.78 H_{Fnd.}: 2.98 N_{Fnd.}: 4.38 I_{Fnd.}: 23.99 F_{Cld.}: 20.16 F_{Fnd.}: 20.45

H₂O(KF): 3.14%

### EXAMPLE 6

6a)

((2-tert-Butoxycarbonylmethoxy-acetyl)-{2,4,6-triiodo-3,5-bis-[methyl-(2,3,4,5,6-pentaacetoxy-hexyl)-carbamoyl]-phenyl}-amino)-acetic Acid-Ethyl Ester

3.6 g (2.39 mmol) of the substance of Example 5c was dissolved in 80 ml of DMF, 0.397 g (2.87 mmol) of K₂CO₃ and 0.32 ml (2.87 mmol) of bromoacetic acid ethyl ester were added, and it was stirred for 4 hours at 40° C. The batch was stirred for another 16 hours at room temperature, then it was filtered, and the clear yellow solution was concentrated by evaporation in a vacuum. The oily crude product was purified by chromatography (EtOAc/hexane 3:1). The yield was 3.61 g (2.27 mmol, 95%) of the title compound. TLC R_{f} 0.42 (EtOAc).

¹H-NMR (CDCl₃): δ (ppm) 1.28 [t, ³J=5.5 Hz, 3H, OCH₂CH₃], 1.46 [m, 9H, OC(CH₃)₃], 2.12 [m, 30H, (O)CCH₃], 2.85 [m, 6H, N(CH₃)CH₂], 3.81 [m, 4H, N(CH₃)CH₂], 4.16 [m, 12H, CH₂OAc, (O)CCH₂OCH₂C(O), NCH₂C(O), OCH₂CH₃], 5.16 [m, 2H, CHOAc], 5.38 [m, 2H, CHOAc], 5.52 [m, 2H, CHOAc], 5.67 [m, 2H, CHOAc]

MS (ESI): m/z (%) 1592 (100) [M+H]⁺, 1536 (25) [M-C₄H₉]⁺

6b)

((2-Carboxylmethoxy-acetyl)-{2,4,6-triiodo-3,5-bis-[methyl-(2,3,4,5,6-pentaacetoxy-hexyl)-carbamoyl]-phenyl}-amino)-acetic Acid-Ethyl Ester

3.61 g (2.27 mmol) of ((2-tert-butoxycarbonylmethoxy-acetyl)-{2,4,6-triiodo-3,5-bis-[methyl-(2,3,4,5,6-pentaacetoxy-hexyl)-carbamoyl]-phenyl}-amino)-acetic acid-ethyl ester (see Example 6a) was dissolved in 40 ml of dichloromethane, and 25 ml of trifluoroacetic acid was added and stirred for 24 hours at room temperature. The solvent was removed in a vacuum, the residue was mixed twice with 50 ml of toluene and in each case evaporated to the dry state. The product was dried in in oil pump vacuum, and 3.49 g (2.27 mmol, 100%) of 8310-1/MS 239 remains. TLC R_{f} 0.09 (EtOAc 100%).

¹H-NMR (CDCl₃): δ (ppm) 1.29 [t, ³J=5.5 Hz, 3H, OCH₂CH₃], 2.11 [m, 30H, O(O)CCH₃], 2.88 [m, 6H, N(CH₃)CH₂], 3.79 [in, 4H, N(CH₃)CH₂], 4.25 [m, 12H, CH₂OAc, (O)CCH₂OCH₂C(O), NCH₂C(O), OCH₂CH₃], 5.15 [m, 2H, CHOAc], 5.37 [m, 2H, CHOAc], 5.51 [m, 2H, CHOAc], 5.68 [m, 2H, CHOAc]

MS (ESI): m/z (%) 1536 (100) [M+H]⁺

0.6c)

({2-[2-Oxo-2-(4-heptadecafluorooctanesulfonyl-piperazin-1-yl)-ethoxy]-acetyl}-{2,4,6-triiodo-3,5-bis-[methyl-(2,3,4,5,6-pentaacetoxy-hexyl)-carbamoyl]-phenyl}-amino)-acetic Acid Ethyl Ester

3.49 g (2.27 mmol) of ((2-carboxylmethoxy-acetyl)-{2,4,6-triiodo-3,5-bis-[methyl-(2,3,4,5,6-pentaacetoxy-hexyl)-carbamoyl]-phenyl}-amino)-acetic acid-ethyl ester (see Example 6b) and 1.55 g (2.73 mmol) of 1-(heptadecafluorooctane-1-sulfonyl)-piperazine (production: WO 97/26017 Example 27a) were dissolved in 100 ml of THF, and the solution was cooled in an ice bath. Then, 0.468 g (2.27 mmol) of DCC was added, and it was stirred for 72 hours at room temperature. Since more starting material could be detected in the TLC, 0.468 g (2.27 mmol) of DCC and in addition 0.263 g (2.27 mmol) of N-hydroxysuccinimide were again added and stirred for another 36 hours. The batch was filtered and concentrated by evaporation, and the crude product was purified by chromatography (EtOAc). TLC R_{f} 0.46 (EtOAc).

¹H-NMR (d₆-DMSO): δ (ppm) 1.22 [m, 3H, OCH₂CH₃], 2.08 [m, 30H, O(O)CCH₃], 2.78 [m, 6H, N(CH₃)CH₂], 3.71 [m, 14H, NCH₂C(O), OCH₂CH₃, piperazine CH₂, N(CH₃)CH₂], 4.11 [m, 4H, CH₂OAc, (O)CCH₂OCH₂C(O)], 4.29 [m, 4H, CH₂OAc, (O)CCH₂OCH₂C(O)], 5.08 [m, 2H, CHOAc], 5.68 [m, 6H, CHOAc]

MS (ESI): m/z (%) 2086 (23) [M+H]⁺

6d)

({2-[2-Oxo-2-[4-(heptadecafluorooctane-1-sulfonyl)-piperazin-1-yl]-ethoxy)-acetyl}-{2,4,6-triiodo-3,5-bis-8methyl-(2,3,4,5,6-pentahydroxy-hexyl)-carbamoyl]-phenyl}-amino)-acetic Acid

2.65 g (1.27 mmol) of ({2-[2-oxo-2-(4-hepadecafluorooctanesulfonyl-piperazin-1-yl)-ethoxy]-acetyl}-{2,4,6-triiodo-3,5-bis-[methyl-(2,3,4,5,6-pentaacetoxy-hexyl)-carbamoyl]-phenyl}-amino)-acetic acid ethyl ester was dissolved in 20 ml of MeOH and then mixed with 7.62 ml (15.24 mmol) of 2N NaOH. The batch was stirred for 24 hours at room temperature, neutralized with dilute hydrochloric acid and diluted to a volume of 150 ml. The solution was again membrane-filtered (Millipore Company, nitrocellulose 0.45 μm, Lot No. H9PM63616). Further purification was carried out by ultrafiltration to a conductivity <10 μS (Millipore Company, regenerated cellulose, Filter Code YM3, NMWL 3000, Lot No. KOCNK709). After the retenetate is freeze-dried, 1.47 g (0.9 mmol, 71%) of the title compound remains as a colorless solid.

MS (ESI): m/z (%) 1638 (33) [M+H]⁺

C_{Cld.}: 29.34 H_{Cld.}: 2.89 N_{Cld.}: 4.28 I_{Cld.}: 23.25 C_{Fnd.}: 27.72 H_{Fnd.}: 3.08 N_{Fnd.}: 4.43 I_{Fnd.}: 21.89 F_{Cld.}: 19.72 F_{Fnd.}: 19.42

H₂O(KF): 3.62%

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German Patent Appln. No. 102 09 018.1, filed Feb. 28, 2002, and U.S. Provisional Application No. 60/363,879, filed Mar. 14, 2002 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula I 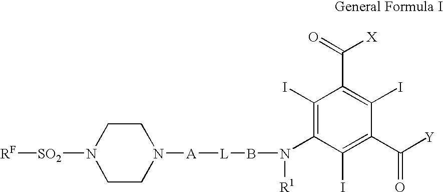
in which:
R^{F} means a straight-chain or branched carbon chain with formula —CₙF₂ₙG, in which G stands for a terminal fluorine, chlorine, iodine or hydrogen atom, and n stands for numbers 4-30.
A and B
A and B are either like or unlike groupings and mean a carbonyl group or sulfonyl group,
L means a straight-chain, branched, saturated or unsaturated carbon chain with 1-15 C atoms, which can be interrupted by 1-5 oxygen atoms, 1-3 sulfur atoms, 1-5 sulfonyl groups, and the latter can optionally be substituted with 1-6 hydroxy groups or 1-3 —(CH₂)ₚ—CO₂H groups, whereby p stands for numbers from 0-10.
R¹ means a hydrogen atom or the group —CH₂)_{q}—CO₂H, whereby q stands for numbers from 1-10.
In addition,
X represents OH, —O⁻Na⁺, —O⁻meglumine⁺ or NR²R³,
Y represents OH, —O⁻Na⁺, —O⁻meglumine⁺or NR⁴R⁵,
whereby
R², R³, R⁴, R⁵ can be the same or different from one another, and represent hydrogen, a straight-chain or branched C₁-C₂₀-alkyl group, whereby the alkyl group can be interrupted by 1-6 oxygen atoms and/or substituted by 1-6 hydroxy groups, or radicals R² and R³ as well as R⁴ and R⁵ with the nitrogen atom to which they are bonded form a heterocyclic ring, which optionally can be substituted with 1-3 hydroxy groups.

2. Compounds according to claim 1, characterized in that G in formula —CₙF₂ₙG means a fluorine atom.

3. Compounds according to claim 1, wherein n in formula —CₙF₂ₙG stands for numbers 4-15.

4. Compounds according to one of claims 1-3, wherein both A and B mean a carbonyl group.

5. Compounds according to one of claims 1-4, wherein L has the following meanings:
—CH₂CH₂—
—CH₂CH₂CH₂—
—CH₂—O—CH₂—
—CH₂CH(OH)CH₂—
—CH₂—O—CH₂CH₂—O—CH₂—

6. Compounds according to one of claims 1-5, wherein R¹ means —H or CH₂COOH.

7. Compounds according to one of claims 1-6, wherein X and Y are identical and mean the following groups:
—N[CH₂—CH(OH)—CH₂OH]₂—
—NH—CH₂—CH(OH)—CH₂OH
—NH—CH(CH₂OH)₂—
—N(CH₃)—CH₂—CH(OH)—CH(OH)—CH(OH)—CH(OH)—CH₂OH
—NH—CH₂—CH(OH)—CH(OH)—CH(OH)—CH(OH)—CH₂OH

8. [{3,5-Bis-[bis-(2,3-dihydroxy-propyl)-carbamoyl]-2,4,6-triiodo-phenyl}-(2-{2-[4-(heptadecafluorooctane-1-sulfonyl)piperazin-1-yl]-2-oxo-ethoxy}-acetyl)-amino]-acetic acid,
N,N,N′N′-tetrakis-(2,3-dihydroxy-propyl)-5-(2-{2-[4-(heptadecafluorooctane-1-sulfonyl)-piperazin-1-yl]-2-oxo-ethoxy}-acetylamino)-2,4,6-triiodo-isophthalamide,
[{3,5-bis-[bis-(2,2-dimethyl-[1,3]dioxolan-4-ylmethyl)-carbamoyl]-2,4,6-triiodo-phenyl}-{4[4-(heptadecafluorooctane-1-sulfonyl)piperazin-1-yl]-4-oxo-butyryl}-amino)-acetic acid-tert-butyl ester,
N,N,N′,N′-tetrakis-(2,3-dihydroxy-propyl)-5-{4-[4-(heptadecafluorooctane-1-sulfonyl)-piperazin-1-yl]-4-oxo-butyrylamino}-2,4,6-triiodo-isophthalamide,
2,4,6-triiodo-N,N′-dimethyl-5-{2-[2-oxo-2-(4-(heptadecafluorooctane-1-sulfonyl)-piperazin-1-yl]-ethoxy]-acetylamino}-N,N′-bis-(2,3,4,5,6-pentahydroxyhexyl)-isophthalamide,
({2-[2-oxo-2-[4-(heptadecafluorooctane-1-sulfonyl)-piperazin-1-yl]-ethoxy)-acetyl}-{2,4,6-triiodo-3,5-bis-8methyl-(2,3,4,5,6-pentahydroxy-hexyl)-carbamoyl]-phenyl}-amino)-acetic acid.

9. Diagnostic agent that contains at least one physiologically compatible compound according to claims 1-8 and optionally physiologically compatible adjuvants that are commonly used in galenicals.

10. Use of at least one physiologically compatible compound according to claims 1-8 as contrast media in x-ray diagnosis.

11. Use of at least one physiologically compatible compound according to claims 1-8 as contrast media for ¹⁹F-NMR diagnosis or ¹⁹F-NMR spectroscopy.

12. Use according to claims 10 and 11 for combined x-ray and NMR diagnosis.

13. Use according to one of claims 9, 10, or 11 for visualizing the intravascular space, the liver, the bile duct, the gastrointestinal tract or the lymph tract.

14. Use of at least one physiologically compatible compound according to claims 1 to 7 as contrast media in ultrasound diagnosis.

15. Use according to one of claims 9 to 13 of at least one physiologically compatible compound according to claims 1 to 7 together with contrast media based on heavy metal chelates.

16. Process for the production of compounds of general formula I according to claim 1, wherein 5-amino-(2,4,5-triiodo)-isophthalic acid chloride (II) is reacted with a dicarboxylic acid-monochloride of general formula III 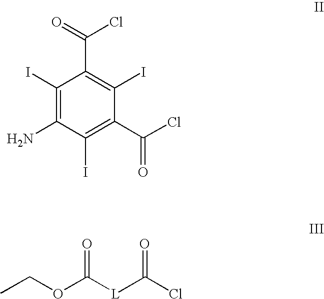
in which L has the meaning mentioned in claim 1, in compounds of general formula IV, in which R¹ and L have the meanings that are mentioned in claim 1, 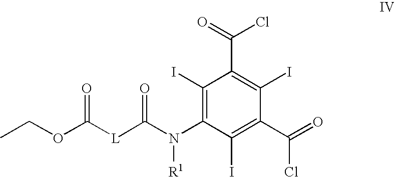
and then IV is reacted with amines of general formula V,
HN—R²R³,
in which R² and R³ have the meanings that are mentioned in claim 1, in compounds of general formula VI 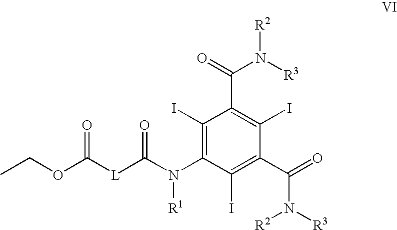
in which R¹, R², R³ and L have the meanings that are mentioned in claim 1, by amide formation reaction, in addition the ethyl ester is saponified in the free acid and further reacted with piperazine derivatives of general formula VII 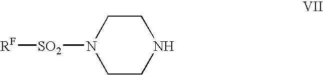
in which R^{F}, which has the meaning that is mentioned in claim 1, is reacted in compounds of general formula VIII 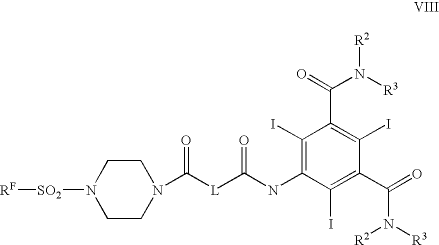
in which R^{F}, R¹, R², R³ and L have the meanings that are mentioned in claim 1, by amide formation, and optionally after cleavage of protective groups, the compounds of general formula I according to the invention are obtained.

17. Process for the production of compounds of general formula I according to claim 1, whereby radical R¹ has the meaning of —CH₂COOH or —CH₂CH₂COOH, the reaction of compounds of general formula VI 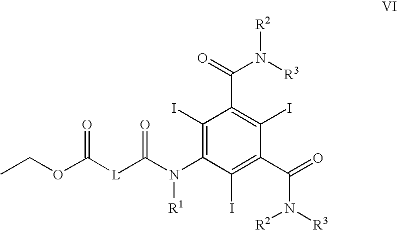
with bromoacetic acid-tert-butyl ester or 2-bromopropionic acid-tert-butyl ester to form compounds of general formula IX, 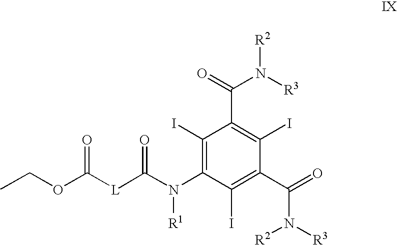
after subsequent cleavage of the ethyl ester in IX, it is reacted to introduce a perfluorinated radical with an amine of general formula VII, 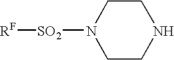
and then the protective groups are cleaved, to obtain the compounds of general formula I with radicals R¹, R² and R³ that are mentioned in claim 1.
